(19) United States
(12) Patent Application Publication
(10) Pub. No.: US 2007/0299364 A1
(43) Pub. Date: Dec. 27, 2007

(54) Containerized sample collection apparatus and method

(76) Inventor: Jangbir S. Sangha, Overland Park, KS (US)

Correspondence Address: SHUGHART THOMSON & KILROY, PC, 120 WEST 12TH STREET, KANSAS CITY, MO, 64105, US

(21) Appl. No.: 11/699,807

(22) Filed: Jan. 30, 2007

Related U.S. Application Data
(60) Provisional application No. 60/815,801, filed on Jun. 22, 2006.

(51) Int. Cl. A61F 13/38

(52) U.S. Cl. 600/572

(57) ABSTRACT

A containerized low pressure sample collection apparatus includes an elongated tubular shaft with a vacuum connection at one end and a swab at an opposite end. The shaft extends through a housing which can be used as a handle. The housing is adapted to receive a cap at either end, at one end for storage and at the other to cover and protect the swab with a collected sample. A second slide-out of the apparatus embodiment includes a swab structure including a tubular shaft, a swab, and a centering structure which can be slid out of and back into a housing which can also be used as a handle to manipulate the apparatus to collect a sample. Both embodiments can be connected to a vacuum unit to draw air through the swab to collect certain kinds of samples.

### CROSS-REFERENCE TO RELATED APPLICATION

This application claims priority under 35 U.S.C. 119(e) and 37 C.F.R. 1.78(a)(4) based upon copending U.S. Provisional Application Ser. No. 60/815,801 for CONTAINERIZED LOW PRESSURE COLLECTION DEVICE, filed Jun. 22, 2006, which is incorporated herein by reference.

### BACKGROUND OF THE INVENTION

The present invention relates to the collection of material samples, as for forensic, scientific, or diagnostic purposes, and more particularly, to containerized, low pressure sample collection apparatus for collecting such samples and preserving them from contamination prior to laboratory analysis, that is, such apparatus in which the sample carrying member is enclosed after collection of the sample to protect the sample from contamination.

Crime scene evidence is collected to establish facts related to a crime or a suspected crime and for identification and/or elimination of suspects and may be presented at a trial for the determination of guilt or innocence of accused individuals. Often, the evidence includes objects, documents, fingerprints, photographs of the scene, and the like. Additionally, the evidence may include unknown substances or substances with a suspected identity, where the identity needs to be determined or confirmed. Such substances may be very small in quantity, may be dispersed over a comparatively large area, and may include materials such as: body fluids, hairs, flakes of skin, fibers, drugs, various chemicals, gunpowder residue, flammable materials, tobacco ashes, cosmetics, and the like. Such materials may be collected at a scene and subjected to chemical and/or DNA analysis for identification or for association with a particular individual.

For collecting substance samples, investigators typically use fibrous swabs, such as swabs made of fibers of cotton, cellulose, rayon, polyester, and other types of fibers. Such swabs not only absorb liquids and solids entrained in liquids but also trap dry substances such as particulate materials. The swabs are kept in closed bags or containers prior to use to maintain sterility and are replaced in such containers after use to avoid contamination of the sample gathered. After replacement of a swab in a container, the container is usually marked with a time and date and identity of the investigator and other information to establish a chain of custody of the sample.

Conventional swabs are formed of a “stick” such as a shaft of wood, tubular plastic, or tubular or rolled paper with a pad of cotton or other fiber, sponge material, or other absorbent material attached to the end of the shaft, either mechanically or by an inert adhesive. A problem with conventional swabs is that there is a danger of contamination of the sample if it is necessary to put the swab down, for example, to open a bag or container in which the swab will be placed. Also, if it is necessary to set the swab down to dry, in a propped up condition or extending over the edge of a table, there is a risk of contamination of the sample.

### SUMMARY OF THE INVENTION

The present invention provides a greatly improved sample or specimen collection apparatus and a low pressure sample collection apparatus. Generally the sample collection apparatus includes an elongated tubular shaft having a swab member secured to a swab end thereof. In one embodiment air is drawn through the shaft from the swab end toward an opposite vacuum connection end of the shaft flows through the swab. A housing is positioned between the ends of the shaft and forms a handle to enable manipulation of the apparatus to collect a sample of a substance. Preferably, the apparatus includes a cap member which can be connected to the housing in covering relation to the swab member to protect it from contamination and which can be stored on an opposite end of the housing. The cap may have one or more holes or apertures formed in its side wall to admit air to dry the swab member when the cap covers the swab member. The cap may also have a desiccant positioned therein to facilitate drying the swab member or the desiccant may be positioned in the tube bottom (FIG. 10).

Some part of the sample collection apparatus is marked with unique identification indicia to distinguish one apparatus from another. Preferably, such identification indicia is applied to the housing. The indicia can be in the form of a barcode, a serial number, or the like. The housing may also have a writable surface, such as a paper tag adhered thereto so that an investigator or technician may write identifying notes on the apparatus, such as initials, a date, a time, a case number, or the like. The apparatus may have a push-off barrel telescoped on the tubular shaft between the housing and the swab member. The barrel is grasped and pushed against the swab member to separate the swab member from the shaft without touching, and possibly contaminating a sample on the swab member, for example for laboratory analysis. Alternatively, the shaft can be scored or be made of a breakable material to allow separation of the swab when the swab is pressed against a side wall of a tube.

The housing may have any cross-sectional shape. A non-round shape resists rolling if the apparatus is placed on a surface. The cross-sectional shape may be rectangular, square, circular, or any other suitable shape. Preferably, ends of the housing have reduced cross-sectional areas to form plugs to receive an open end of the cap member. The sample collection apparatus may be used to collect liquid or dry samples. A portable, low pressure vacuum unit may be connected to the vacuum connection end of the shaft to establish inward air flow through the swab member to facilitate collection of dry samples, such as particulate or flaked materials. Alternatively, the sample collection apparatus may be used without such a vacuum unit.

A modified slide-out embodiment of the sample collection apparatus provides a swab assembly which can be retracted into a housing to protect a swab member from contamination. The apparatus includes an elongated tubular shaft having a swab member secured to a swab end and has an opposite vacuum connection end. The shaft is slidably mounted through a tubular housing member by means of a centering structure which maintains the swab member out of contact with walls of the housing when retracted therein. The housing has flap members at a swab end thereof which can be closed to protect the swab member from contamination. The shaft, swab member, and centering structure form a swab assembly which is slidable through the housing. The swab assembly is advanced to an extended position to extend the swab member out of the housing to collect a sample. The swab assembly is retracted to retract the swab member within the housing, after which the flaps may be closed to protect a sample collected on the swab member from contamination. The shaft may include a push-off barrel positioned between the centering structure and the swab member. The barrel can be used to separate the swab member from the shaft without touching the swab member. The housing is preferably provided with unique identification indicia, such as a barcode, serial number, or the like. Additionally, the housing may be provided with a writable surface to receive notes from an investigator or technician.

Also taught is a method of forensic specimen collection which provides for a specimen collector having a unique barcode thereon within a clear plastic wrapper; the wrapper having multiple copies of the identical unique barcode which is on the collector. The clear packaging permits the crime scene investigator to photograph the unopened collector next to the specimen to be collected, thus recording the series of barcodes adjacent the specimen prior to collection. The additional barcodes allow the investigator to use the additional barcodes to cross-reference the specimen with notes in a notebook and to cross-reference a tamper-proof seal applied to the specimen container after specimen collection thereon, and to cross-reference any transportation packaging used to ship the collector having the specimen thereon to a laboratory for analysis.

Objects and advantages of this invention will become apparent from the following description taken in conjunction with the accompanying drawings wherein are set forth, by way of illustration and example, certain embodiments of this invention.

### BRIEF DESCRIPTION OF THE DRAWINGS

The drawings constitute a part of this specification and include exemplary embodiments of the present invention and illustrate various objects and features thereof.

FIG. 1 is a perspective view of a sample collection apparatus which is positioned in a sealable bag;

FIG. 2 is a perspective view of the sample collection apparatus removed from the bag of FIG. 1 for use in the field to collect a sample of a substance;

FIG. 3 is a perspective view of the sample collection apparatus with a cap removed to expose a vacuum connection, the cap being positioned to cover a swab member after a sample has been collected;

FIG. 4 is a perspective view of the sample collection apparatus with the cap in covering relation to the swab member and diagrammatically showing an aperture in the cap to facilitate drying of the swab member;

FIG. 5 is a perspective view of a slide-out embodiment of the sample collection apparatus shown positioned in a sealable bag;

FIG. 6 is a perspective view of the slide-out sample collection apparatus, shown with a swab member extended for use in the field to collect a sample of a substance;

FIG. 7 is a perspective view of the slide-out sample collection apparatus, shown with the swab member being retracted into an integral housing of the apparatus;

FIG. 8 is a perspective view of a modified embodiment of the sample collection apparatus which has a cylindrical cross section, shown with a cap removed to expose a vacuum connection and with the cap positioned to be placed over a swab member thereof;

FIG. 9 is a perspective view of the cylindrical sample collection apparatus, shown with the cap in covering relation to the swab and diagrammatically showing a desiccant positioned in the cap;

FIG. 10 shows a schematic representation of method steps related to a crime scene method of investigation in which a collector within a clear plastic bag and having a unique indicia thereon and additional copies of the unique indicia on the bag placed next to a crime specimen to be sampled and showing photographic recording of the juxtaposition between the sample and the unopened bag and showing the photograph recording the juxtaposition and the relevant barcodes with additional representation showing the barcodes transferred from the bag to a crime investigator's notebook and to a transportation bag for shipping the collector having a specimen collected thereon and showing a copy of the unique barcode indicia applied to a tamper-proof tape that has been applied to the specimen collector after collecting the sample and sealing the absorbent collector into the housing or tube of the collector;

FIG. 11 shows a collector being applied to a crime scene specimen for collection of a specimen onto the absorbent of the collector;

FIG. 12 shows the absorbent collector having the specimen adhered to the absorbent being inserted into the container or housing used to protect the absorbent from cross-contamination after specimen collection;

FIG. 13 shows a specimen collector having a scored shaft line adjacent the collection absorbent;

FIG. 14 shows the collector absorbent of the collector of FIG. 13 being pressed against the sidewall of a microspecimen vial to separate the absorbent along the score line; and

FIG. 15 shows the absorbent of FIGS. 13 and 14 now separated from the shaft of the collector of FIGS. 13 and 14 and with the absorbent residing within the microspecimen container for analysis of the specimen and with a line indicating transfer of the unique barcode from the container of FIG. 13 onto the micro-vial of FIG. 15 for maintenance of the chain of custody.

### DETAILED DESCRIPTION OF THE INVENTION

As required, detailed embodiments of the present invention are disclosed herein; however, it is to be understood that the disclosed embodiments are merely exemplary of the invention, which may be embodied in various forms. Therefore, specific structural and functional details disclosed herein are not to be interpreted as limiting, but merely as a basis for the claims and as a representative basis for teaching one skilled in the art to variously employ the present invention in virtually any appropriately detailed structure.

Referring now to the drawing figures, the reference numeral 10 generally designates a containerized low pressure sample collection apparatus. The apparatus 10 generally includes a swab assembly 11 (FIG. 3) which extends through a housing structure 18 which may function as a handle to manipulate the apparatus 10.

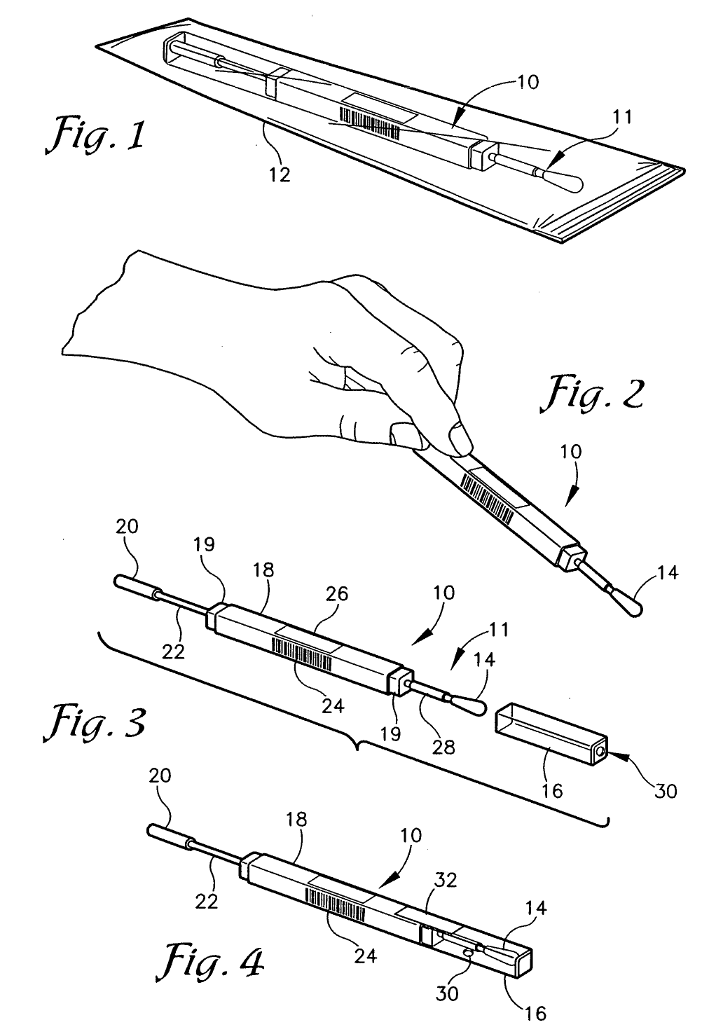

Referring to FIGS. 1-4, the illustrated swab assembly 11 includes an elongated tubular shaft 22 having a vacuum connection 20 at a vacuum connection end thereof and an absorbent swab member 14 secured to a swab end of the shaft 22. The shaft 22 extends through the housing structure 18 and is secured thereto, as by friction, an adhesive, welding, or the like. The illustrated housing 18 has sections of reduced cross-section at its ends to form plug members 19 to receive a cap member 16 on either end of the housing. The cap 16 may be placed on the vacuum connection end for storage of the cap, as shown within a sealable bag or container 12 in FIG. 1, or on a swab end of he housing 18 to protect the swab member 14, as shown in FIG. 4. The cap member 16 may be provided with an opening or aperture 30 in a side wall or an end wall to admit air for drying the swab member 14 when enclosed therein. The apparatus 10 may be provided with a push-off barrel 28 (FIG. 3) which is coaxially mounted on the shaft 22 between the housing 18 and the swab member 14. The barrel 28 may be pushed against the swab member 14 to separate it from the shaft 22 without otherwise touching the swab to prevent contamination of a sample thereon.

The swab member 14 may be provided in various forms and of various materials depending on the suspected identity and the character of the material to be sampled. The sampled material may be a liquid, a liquid with suspended solids, a gel or a grease, a particulate or granular material, a flaked material, and so forth. Thus, the swab member 14 may be formed of a fibrous material, such as cotton, cellulose, rayon, polyester, or the like, various kinds of sponge or porous materials, a fabric, a paper, a netting material, or the like. The swab member 14 may be in a sterile condition or it may be coated or impregnated with a chemical to attract or bind with a particular substance or coated to prevent migration of the sampled material too deeply into the swab member 14.

The vacuum connection 20 provides for the connection of a portable, low pressure vacuum unit (not shown) thereto to cause an air flow through the swab member 14 into the tubular shaft 22. Such an air flow can facilitate collection of certain kinds of materials within the swab member 14. Alternatively, the apparatus 10 can be used without such a vacuum source.

To establish and maintain a chain of custody of a sample collected using the apparatus 10, it may be marked with identification indicia which is unique to the particular apparatus 10. As illustrated, the housing 18 is marked with indicia 24, such as a barcode. Alternatively, such indicia 24 could be in the form of an alphanumeric serial number. However, an advantage of a barcode is that it can be quickly and accurately scanned, while a serial number is vulnerable to copying errors and transposition of characters. Preferably, no two apparatus 10 have the same indicia 24 so than once the indicia 24 of an apparatus 10 is recorded and associated with a particular case, another apparatus 10 cannot be substituted for the recorded one. The apparatus 10 may be provided with a writable surface 26 to record short notes, such as the initials of the investigator or technician, a time or date, a case number, or the like. The writable surface 26 may be provided as a paper tag adhered to the surface of the housing 18. The apparatus 10 may also be provided with a tamper indicator 32, such as an adhesive seal adhered to the housing 18 and the cap 16 after a sample is collected on the swab member 14.

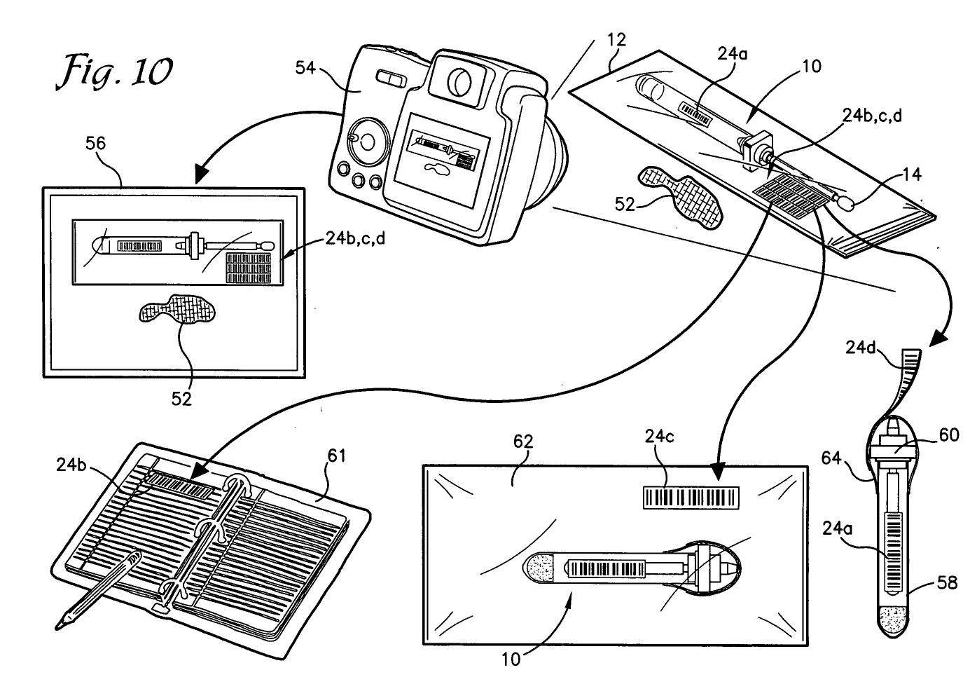

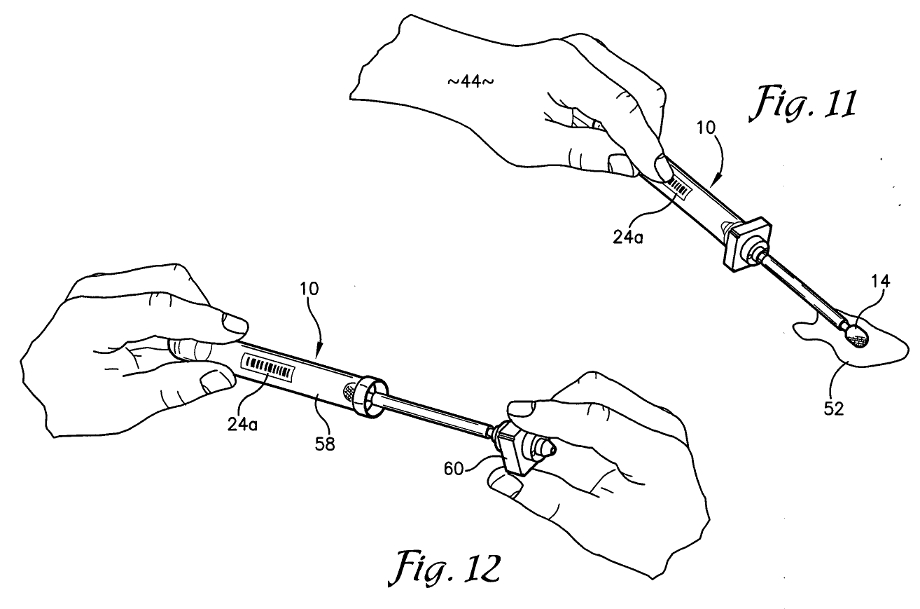

The identifying indicia 24 may be a unique indicia that is assigned to the collector 10 at the time of manufacture of the collector 10. The unique indicia 24 is intended to be sufficient to distinguish any one collector 10 from all other such collectors ever manufactured. The importance of providing such a unique indicia 24 is that the collector 10 is to be used at a crime scene and may be photographed being used at the crime scene during the evidence collection process (See, FIG. 10), the photographing thus serving to verify the use and association of one particular, unique collector 10 in the collection of evidence at the crime scene. Having a particular indicia uniquely assigned to one and only one device 10 is necessary to provide confirmation that substitute devices were not, and could not have been introduced into the evidence gathering. This uniqueness of identification of device 10 makes concrete the chain of custody of the crime evidence collected on the device 10 and is necessary to eliminate claims that a mix-up in devices 10 or a substitution of devices 10 having the same identification indicia may have occurred thereby invalidating the evidence collection. In this manner, a law enforcement agency can ensure and demonstrate that a device 10 presenting a particular unique indicia that appears in a photograph taken at a crime scene is the only such device in existence, and that no second device 10 that by chance has the same identification number has been inadvertently substituted for the particular device 10 actually used and photographed at the crime scene to collect the evidence being submitted in a prosecution.

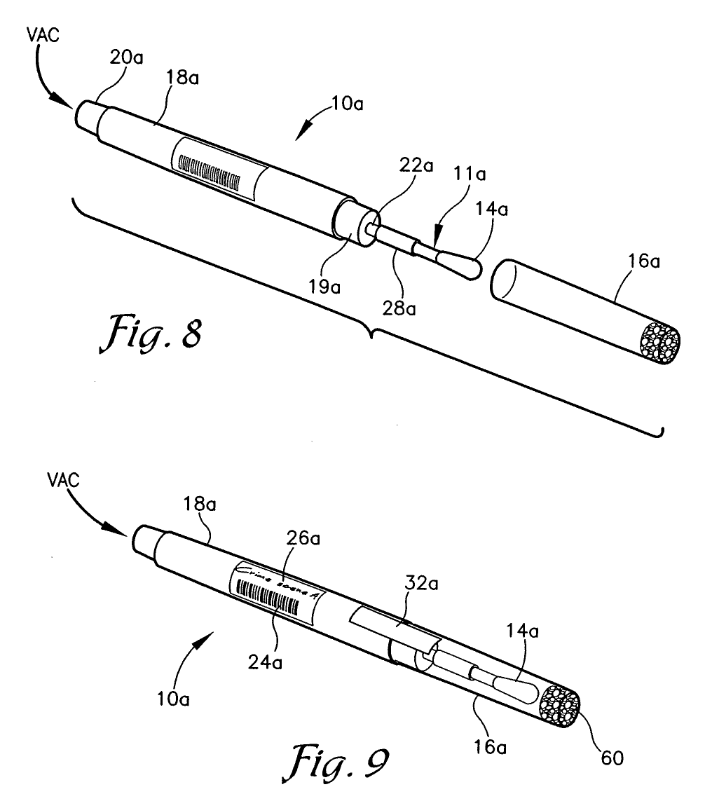

The apparatus 10 is illustrated in FIGS. 1-4 with a housing 18 having a substantially square cross-sectional shape. Alternatively, the housing 18 could have other non-round cross-sectional shapes, such as rectangular, triangular, elliptical, or the like. FIGS. 8 and 9 illustrate a modified embodiment 10a of the containerized low pressure sample collection apparatus. The apparatus 10a includes a swab assembly 11a formed by an elongated tubular shaft 22a extending through a housing structure 18a and terminating at a vacuum connection end with a vacuum connection 20a. The illustrated housing 18a has a circular cross-sectional shape and is, thus, cylindrical. At an end opposite the vacuum connection 20a, a swab member 14a is secured to the shaft 22a. The housing structure 18a may be used as a handle for manipulating the apparatus 10a to collect a sample of a substance on the swab member 14a. The housing 18a may be provided with a section of reduced diameter which forms a plug member 19a to receive the open end of a cap member 16a to cover the swab member 14 after a sample has been collected. The illustrated vacuum connection 20a may also be sized to receive the end of the cap 16a for storage thereof. The vacuum connection 20a provides for connection of a low pressure vacuum unit (not shown) to draw air through the swab member 14a to facilitate collection of samples of certain kinds of materials. Alternatively, the apparatus 10a may be used for collection of other types of materials without the use of such a vacuum unit.

As shown in FIG. 9, a tamper seal 32a may be provided to adhere to the housing 18a and the cap 16a to show visible signs of attempts to remove the cap 16a. The housing 18a may be provided with identification indicia 24a, such as a barcode which uniquely identifies the apparatus 10a. The housing 18a may, further, be provided with a writable area 26a to receive written notes pertaining to the sample collected. The illustrated apparatus 10a has a push-off barrel 28a positioned on the shaft 22a between the housing 18a and the swab member 14a for use in pushing the swab member 14a off the end of the shaft 22a. The cap 16a may have a quantity of a desiccant material 60 positioned therein to facilitate drying of the swab member 14a when covered by the cap 16a. Alternatively, the cap 16a may be provided with an opening (not shown) similar to the opening 30 in the cap 16 of FIGS. 1-4. It should be noted that the cap 16 in FIGS. 3 and 4 could also be provided with a desiccant material 60 to promote drying of the swab member 14.

FIGS. 5-7 illustrate a slide-out embodiment 10b of the containerized low pressure sample collection apparatus. The apparatus 10b includes a swab assembly 11b which is slidably supported by a hollow or tubular housing 40, which forms an enclosure for a swab member 14b or which can function as a handle for manipulating the apparatus 10b to collect a sample. The swab assembly 11b includes an elongated tubular shaft 22b having a vacuum connection 20b at a vacuum connection end and the swab member 14b secured at an opposite swab end. The swab assembly 11b includes a swab support and centering structure 50 formed by panels 52 and 54 and additional box structure members (not shown) within the housing 40. The swab assembly 10b may include a push-off barrel 28b telescoped onto the shaft 22b between the support structure 50 and the swab member 14b for use in separating the swab member 14b from the shaft 22b. The swab member 14b, the support structure 50, the portion of the shaft 22b therebetween, and the barrel 28b, if present, form a sample collection head 46 of the swab assembly 11b. The vacuum connection 20b allows a portable low pressure vacuum source (not shown) to be connected to the apparatus 10b to establish an air flow through the swab member 14b and the shaft 22b to facilitate collection of some types of samples. Alternatively, the apparatus 10b may be used without such a vacuum source.

The illustrated housing 40 is an elongated structure with a square cross-section through which the swab assembly 11b extends. At a swab end 42 (FIG. 5) toward the swab member 14b, the housing 40 has a pair of flaps 48a and 48b which cooperate to close the swab end of the housing 40. The housing 40 may be formed from a material such as a stiff paper, a plastic, or a composite of sheet materials. The apparatus 10 may be provided in a sealable bag or container 12b and may be replaced in the bag 12b after use.

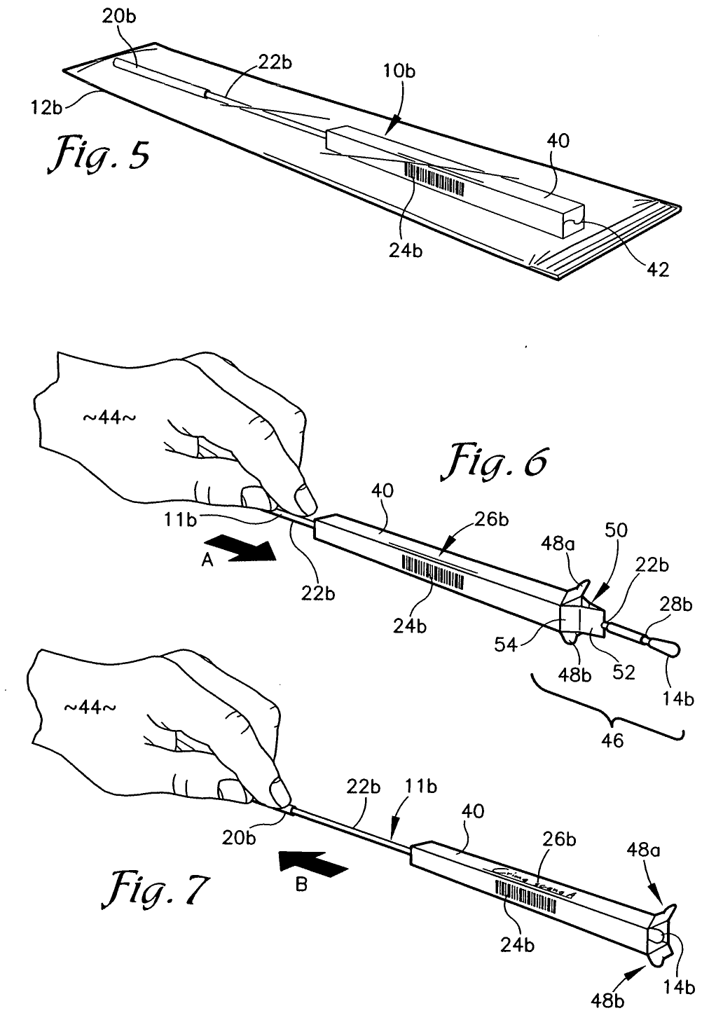

The sample collection apparatus 10b would typically be provided in the bag 12b with the sample collection head 46 retracted within the housing 40 and the flaps 48a and 48b closed to prevent contamination of the swab member 14b. For use in the field to collect a sample of a substance, the apparatus 10b is removed from the bag 12b, the portion of the shaft 22b near the vacuum connection 20b is grasped in one hand 44, the housing 40 grasped in the other hand, and the swab assembly 11b advanced in the direction of arrow A (FIG. 6) to extend the sample collection head 46 out of the housing 40 through the flaps 48a and 48b. A source of vacuum can be connected to the vacuum connection 20b if suitable. The housing 40 may be used as a handle to manipulate the apparatus 10b in collecting the sample.

After the sample is collected, the vacuum connection end of the swab assembly 11b is again grasped and pulled in the direction of arrow B (FIG. 7) to retract the sample collection head 46 within the housing 40 for protection against contamination of the sample. The flaps 48a and 48b can then be closed. It is foreseen that a tamper seal, similar to the tamper seal 32 of FIG. 4, could be adhesively applied to the flaps 48a and 48b to indicate any attempt to access the swab member 14b prior to official analysis. The swab support and centering structure 50 retains the swab member 14b out of contact with the walls of the housing 40 when the swab member 14b is positioned within the housing 40. After the swab assembly 11b is retracted and the flaps 48a and 48b closed, the apparatus 10b may be replaced in the bag 12b for transport to a laboratory for analysis of the sample collected. It will be appreciated by those skilled in the art that after a liquid specimen has been collected on swab member 14b that drying of the liquid specimen is preferred. In the present embodiment swab support and centering structure 50 retains the swab member 14b in an elevated position while collection apparatus 10b is resting on a surface thereby avoiding cross-contamination of swab member 14b and the specimen collected thereon.

The illustrated housing 40 is with identifying indicia 24b, such as a barcode, to uniquely identify the sample collection apparatus 10b. Alternatively, other types of identifying indicia could be provided, such as a serial number or the like. The housing 40 may also be provide with a writable surface 26b to allow an investigator or technician to record selected notes on the housing 40.

Referring now to FIG. 10, the method of forensic specimen collection while maintaining a chain of custody, will be described. As previously described, it is important for a law enforcement agency to ensure and demonstrate that any specimen collection device 10 has been properly maintained within police custody and that tampering with the specimen has been prevented or that any attempt to tamper with the specimen would be indicated by some type of physical modification of the specimen or the container within which the specimen is held. Further, in maintaining the chain of custody of crime scene evidence and providing sufficient proof that laboratory analyses were performed on a particular specimen actually obtained from a crime scene, it is necessary to document the entire collection process, transportation process, analysis process and reporting of results for any crime scene specimen that is collected. The present apparatus may be used in a method of evidence collection which, by the structure of the apparatus, permits use of a method which serves to maintain the chain of custody of crime scene evidence and to assist in the documentation of the collection and analysis of the evidence.

Still referring to FIG. 10, a crime scene specimen 52, is shown which may be a fluid such as blood or saliva, or the specimen 52 may be a solid which has dried at a crime scene or the specimen 52 may be any other unknown piece of evidence which is collectable by contacting the specimen 52 with a swab member 14, such as that shown attached to collector 10.

In the method of evidence collection, a crime scene investigator places sealable bag or container 12 having apparatus 10 therein adjacent a crime scene specimen 52. The investigator then uses a camera 54 to photograph the unopened sealable bag 12 having apparatus 10 inside adjacent a crime scene sample specimen 52. The result of this step is that a photograph 56 is generated which documents the particular specimen 52 to be collected adjacent the collector 10 still sealed with bag 12 and bearing unique indicia 24a on collector 10 and indicia 24b,c,d in place on the bag. This serves to verify that prior to collection, container 10 was in bag 12 and in good condition and that bag 12 had not been opened to allow prior access container 10 and swab 14.

The step of sample collection is shown in FIG. 11, wherein container 10 has been removed from bag 12 (FIG. 10) and swab member 14 is applied to specimen 52, such that a portion specimen 52 adheres to swab member 14. Once the portion of specimen 52 has been collected on swab member 14, the swab member 14 is inserted into container 58 and swab member 14 is sealed within container 58 by the frictional fit between container 58 and cap 60. It will be appreciated that indicia 24a continues to be present on container 58 to maintain the particular, unique identity of container 58.

Again referring to FIG. 10, it may be desirable for the crime scene investigator to record various observations and notations regarding specimen 52 and the manner of collection of specimen 52 in a notebook 61. To allow the crime scene investigator to cross-reference, specimen 52 and collector 10, with the notes taken in notebook 61, a second copy of unique indicia 24, in this case, 24b, is removed from bag 12 and applied to notebook 61. It will be appreciated that indicia 24a is adhered at the time of manufacture to container 10 and indicia 24b,c,d are adhered to the outside of or inside of bag 12 at the time of manufacture. Once the crime scene investigator has photographed bag 12 and container 10 adjacent to specimen 52, and has recorded the investigator's observations in notebook 61, it is then necessary to transport the container 58 having swab member 14 therein to a laboratory for analysis. This is accomplished by placing collector 10 into a transportation bag 62 to which a third copy of unique indicia 24, in this case unique indicia 24c, is applied to the outside or to the inside of bag 62. Container 10, having been inserted into bag 62 is then sealed within bag 62 such that access to container 10 can only be achieved by some degree of physical destruction of bag 62. Prior to inserting container 10 into bag 62, it is good practice to apply an additional seal to container 58 once cap 60 has been inserted to close container 58. This additional seal is typically in the form of a tape seal such as tamper-proof tape 64, which has been applied to container 58 and to cap 60. It will be appreciated by those skilled in the art that once the tamper-proof tape has been applied to container 58 and cap 60, that entry into tube 58 can only be achieved by the physical destruction of tamper-proof tape 64. To particularly identify tamper-proof tape 64 that has been applied by the investigator at a crime scene, a fourth copy of indicia 24, in this case indicia 24d, is adhered to tamper-proof tape 64. In this manner, the destruction of tamper-proof tape 64, also would involve destruction of unique indicia 24d, which would demonstrate tampering with the container 58 and potentially the sample within on swab member 14. It further will be appreciated that an individual attempting to substitute a new tamper-proof seal for the tamper-proof seal 64 applied by the investigator, would be frustrated by the lack of a copy of unique indicia 24d to apply to the substituted tamper-proof seal 64. This may be the case where the adhesive used to apply unique indicia 24d to tamper-proof 64 is a non-releasable adhesive and the separation of any of unique indicia 24a,b,c,d from the object to which they applied after removal from bag 12, will result in the destruction of the unique indicia thus preventing substitution of the indicia 24a,b,c,d onto an alternate item.

Therefore, as described, it will be appreciated that the method taught herein provides a complete, verifiable, chain of custody which begins at the time just prior to collection of a specimen and continues to the time at which the specimen is received at the laboratory for analysis, while further extending to provide verification of investigator notes in a crime notebook and additional identification and verification of tamper-proof tape used to seal the specimen collector. It further will be appreciated that as unique indicia 24 is intended to be a one, and only one use identifier, that the particular crime scene at which collector 10 is used, will be uniquely identifiable from all other crime scenes and that any notes taken by an investigator in notebook 60, will also be uniquely identifiable in addition to the maintenance to the chain of custody as taught by the maintenance of the chain of custody as taught by the present method.

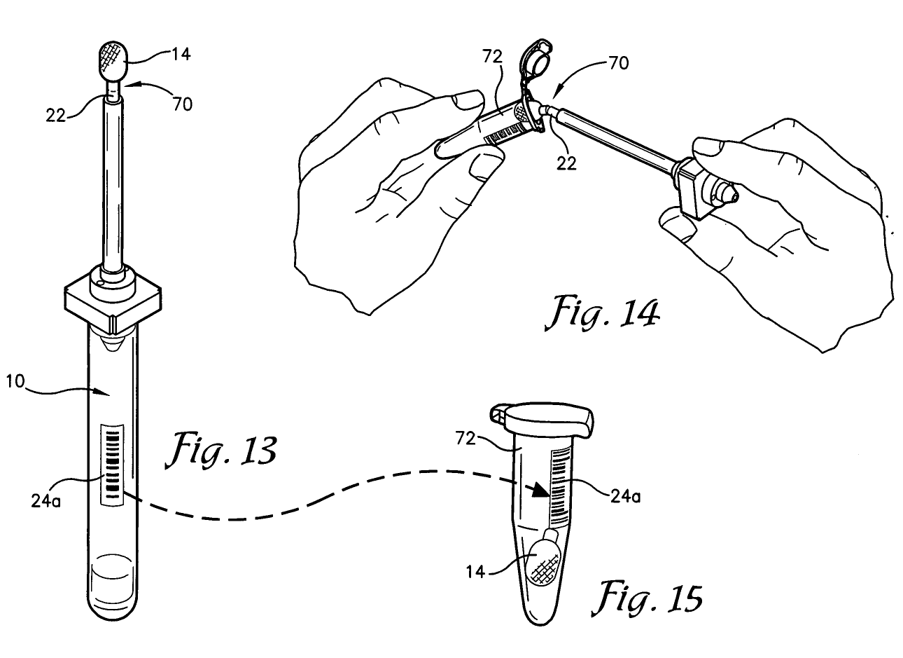

Referring now to FIGS. 13, 14, and 15, an additional embodiment will be described. The embodiment of FIGS. 13, 14, and 15 permits ease of separation of swab member 14 from collector 10 and in particular, the separation of swab member 14 from shaft 22 to allow swab 14 alone to be inserted into a container or vial for storage or analysis or elution of the material collected on swab 14. The ease of separation previously described is achieved by inclusion of a score line 70 on shaft 22. This score line 70, or line of weakness or shaft of fracturable material, permits snapping or breaking of shaft 22 at the point of score line or point of weakness 70. This function is shown in use in FIG. 14 wherein swab 14 is introduced into a vial 72 and by firmly biasing swab 14 against the wall of vial 72, shaft 22 may be caused to break along score line 70, thereby releasing swab 14 from shaft 22 and allowing the depositing of swab 14 within vial 72 as shown in FIG. 15. It will be appreciated that for maintenance of the chain of custody, that barcode 24a may be made transferable thereby permitting transfer of barcode 24a unto vial 72 to thereby maintain the identification of vial 72 and swab 14 contained therein. It will be appreciated by those skilled in the art, that such transfer of indicia 24a is not in keeping with the previously described method of chain of custody, and that this particular embodiment is not intended to detract from the previously described chain of custody.

It is to be understood that while certain forms of the present invention have been illustrated and described herein, it is not to be limited to the specific forms or arrangement of parts described and shown.

What is claimed and desired to be secured by Letters Patent is:

1. A sample collection apparatus for collecting a sample of a substance and comprising:
(a) an elongated tubular shaft having opposite ends including a vacuum connection end and an opposite swab end;
(b) a swab member secured to said swab end of said shaft in such a manner that air drawn through said shaft from said swab end toward said vacuum connection end flows through said swab member; and
(c) a housing positioned between said opposite ends of said shaft and forming a handle to enable manipulation of said apparatus to collect a sample of a substance.

2. A sample collection apparatus as set forth in claim 1 and including:.
(a) a cap member removably engageable with said housing to cover said swab member to prevent contamination of said swab member and any sample substance therein.

3. A sample collection apparatus as set forth in claim 2 and including:
(a) an adhesive tamper seal applied to said housing and said cap member after a sample is taken with said swab member and said cap member is engaged with said housing in covering relation to said swab member to thereby visibly show removal of said cap thereafter.

4. A sample collection apparatus as set forth in claim 2 and including:
(a) a desiccant material positioned within said cap member to facilitate drying of said swab member.

5. A sample collection apparatus as set forth in claim 1 wherein said housing has a vacuum end and a swab end and including:
(a) a vacuum end plug extending from said vacuum end and a swab end plug extending from said swab end; and
(b) a cap member removably engageable with said swab end plug to cover said swab member and engageable with said vacuum end plug for storage of said cap member.

6. A sample collection apparatus as set forth in claim 1 and including:
(a) a cap member removably engageable with said housing to cover said swab member to prevent contamination of said swab member and any sample substance therein; and
(b) said cap member having an opening therein to enable entry of air therein when said cap member is engaged with said housing covering said swab member to thereby facilitate drying of said swab member.

7. A sample collection apparatus as set forth in claim 1 and including:
(a) unique identification indicia marked on said housing to uniquely identify said apparatus.

8. A sample collection apparatus as set forth in claim 1 and including:
(a) a unique barcode marked on said housing to uniquely identify said apparatus.

9. A sample collection apparatus as set forth in claim 1 wherein said housing includes:
(a) a writable surface on said housing to enable selective marking thereof.

10. A sample collection apparatus as set forth in claim 1 wherein:
(a) said housing has a non-round cross-sectional shape.

11. A sample collection apparatus as set forth in claim 1 wherein:
(a) said housing has a rectangular cross-sectional shape.

12. A sample collection apparatus as set forth in claim 1 wherein:
(a) said housing has a substantially square cross-sectional shape.

13. A sample collection apparatus as set forth in claim 1 wherein:
(a) said housing has a substantially cylindrical shape.

14. A sample collection apparatus as set forth in claim 1 wherein:
(a) said housing is slidably supported on said shaft between a retracted position in which said swab member is positioned within said housing and an extended position in which a portion of said shaft with said swab member thereon extends from said housing.

15. A sample collection apparatus as set forth in claim 14 and including:
(a) said housing has a swab end toward said swab member;
(b) said swab end of said housing has a flap member which is closable with said housing in said retracted position to prevent contact with said swab member.

16. A sample collection apparatus as set forth in claim 14 and including:
(a) a swab support structure positioned on said shaft within said housing to position said swab end said shaft with said swab member thereon at a central position within said housing to thereby avoid contact of said swab member with inner surfaces of said housing when positioned therein.

17. A sample collection apparatus as set forth in claim 1 and including:
(a) a push-off barrel positioned on said shaft between said housing and said swab member, said barrel being selectively pushed into engagement with said swab member to separate said swab member from said swab end of said shaft.

18. A sample collection apparatus for collecting a sample of a substance and comprising:
(a) an elongated tubular shaft having opposite ends including a vacuum connection end and an opposite swab end;
(b) a swab member secured to said swab end of said shaft in such a manner that air drawn through said shaft from said swab end toward said vacuum connection end flows through said swab member;
(c) a housing positioned between said opposite ends of said shaft and forming a handle to enable manipulation of said apparatus to collect a sample of a substance, said housing having a vacuum end and a swab end and including:
(1) a vacuum end plug extending from said vacuum end and a swab end plug extending from said swab end; and
(2) a cap member removably engageable with said swab end plug to cover said swab member and engageable with said vacuum end plug for storage of said cap member;
(d) said cap member having an opening therein to enable entry of air therein when said cap member is engaged with said housing covering said swab member to thereby facilitate drying of said swab member;
(e) unique identification indicia marked on said housing to uniquely identify said apparatus; and
(f) a push-off barrel positioned on said shaft between said housing and said swab member, said barrel being selectively pushed into engagement with said swab member to separate said swab member from said swab end of said shaft.

19. A sample collection apparatus as set forth in claim 18 and including:
(a) a desiccant material positioned within said cap member to facilitate drying of said swab member.

20. A sample collection apparatus as set forth in claim 18 wherein:
(a) said unique identification indicia includes a unique barcode marked on said housing to uniquely identify said apparatus.

21. A sample collection apparatus as set forth in claim 18 wherein said housing includes:
(a) a writable surface on said housing to enable selective marking thereof.

22. A sample collection apparatus as set forth in claim 18 wherein:
(a) said housing has a non-round cross-sectional shape.

23. A sample collection apparatus as set forth in claim 18 wherein:
(a) said housing has a substantially square cross-sectional shape.

24. A sample collection apparatus for collecting a sample of a substance and comprising:
(a) an elongated tubular shaft having opposite ends including a vacuum connection end and an opposite swab end;
(b) a swab member secured to said swab end of said shaft in such a manner that air drawn through said shaft from said swab end toward said vacuum connection end flows through said swab member;
(c) a housing positioned between said opposite ends of said shaft and forming a handle to enable manipulation of said apparatus to collect a sample of a substance, said housing being slidably supported on said shaft for sliding movement between a retracted position in which said swab member is positioned within said housing and an extended position in which a portion of said shaft with said swab member thereon extends from said housing;
(d) said housing having a swab end toward said swab member and having a flap member at said swab end which is closable with said housing in said retracted position to prevent contact with said swab member; and
(e) a swab support structure positioned on said shaft within said housing to position said swab end said shaft with said swab member thereon at a central position within said housing to thereby avoid contact of said swab member with inner surfaces of said housing when positioned therein.

25. A sample collection apparatus as set forth in claim 24 and including:
(a) unique identification indicia marked on said housing to uniquely identify said apparatus.

26. A sample collection apparatus as set forth in claim 24 and including:
(a) a unique barcode marked on said housing to uniquely identify said apparatus.

27. A sample collection apparatus as set forth in claim 24 wherein said housing includes:
(a) a writable surface on said housing to enable selective marking thereof.

28. A sample collection apparatus as set forth in claim 24 wherein:
(a) said housing has a substantially square cross-sectional shape.
